(19) United States
(12) Patent Application Publication
(10) Pub. No.: US 2024/0016744 A1
(43) Pub. Date: Jan. 18, 2024

(54) NALTREXONE AND RISPERIDONE COMBINATION SUSTAINED-RELEASE COMPOSITION

(71) Applicant: SHENZHEN SCIENCARE MEDICAL INDUSTRIES CO.,, Shenzhen, Guangdong (CN)

(72) Inventors: Xieguo YAN, Shenzhen (CN); Fuqiao YAN, Shenzhen (CN); Guohui LIU, Shenzhen (CN); Shiqiang WANG, Shenzhen (CN)

(73) Assignee: SHENZHEN SCIENCARE MEDICAL INDUSTRIES CO.,, Shenzhen, Guangdong (CN)

(21) Appl. No.: 18/314,047

(22) PCT Filed: Dec. 10, 2020
(86) PCT No.: PCT/CN2020/135369

§ 371 Date: May 8, 2023

(30) Foreign Application Priority Data
Nov. 9, 2020 (CN) 202011243436.5

(51) Int. Cl. A61K 9/20; A61K 31/485; A61K 31/519; A61K 9/16; A61P 25/30

(52) U.S. Cl. CPC A61K 9/2077; A61K 31/485; A61K 31/519; A61K 9/1647; A61K 9/1635; A61K 9/2013; A61P 25/30

(57) ABSTRACT

The present disclosure relates to a preparation method for a preparation, in particular, to a compound sustained-release composition of naltrexone and risperidone, and a preparation method therefor and an application thereof. The compound sustained-release composition of naltrexone and risperidone and its preparation provided in the present disclosure may be sustainably released in vitro for more than 12 weeks, the release conforms to zeroth-order approximation mode, and the release rate is stable; the suitable preparation method and parameter control are adopted in the present disclosure, so that naltrexone sustained-release microspheres and risperidone sustained-release microspheres are released synchronously, and an administration period is easier to control; further, the product provided by the present disclosure has a good effect in methamphetamine addiction tests, and can be used for preparing products for the treatment of methamphetamine addiction.

### CROSS REFERENCE TO RELATED APPLICATIONS

This application is a U.S. National Stage Application under 35 U.S.C. 371 of International Application PCT/CN2020/135369, filed Dec. 10, 2020, which claims priority to Chinese Application No. 202011243436.5, filed Nov. 9, 2020, the contents of each of which is hereby incorporated by reference in its entirety.

Invention Field

The present disclosure relates to a method for producing a preparation, and specifically, to a compound sustained-release composition of naltrexone and risperidone, and preparation method and application thereof.

### BACKGROUND

Naltrexone is a pure antagonist for opioid receptors, and has a block effect on δ-, and κ-opioid receptors. Naltrexone can block the effect of retaking drugs, thereby weakening the positive reinforcement and negative reinforcement, and playing a good supporting role in prevention of relapse. Naltrexone has the following chemical structure:

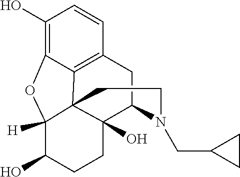 [SMILES: Oc1ccc2c3c1O[C@H]1[C@H](O)CC[C@@]4(O)[C@@H](C2)N(CC2CC2)CCC[C@]314]

Risperidone has a good therapeutic effect on positive and negative symptoms and accompanying affective symptoms thereof (such as anxiety and depression). Risperidone may also reduce affective symptoms associated with schizophrenia. For patients who benefit from treatment during the acute period, this product can continue to exert its clinical efficacy during the maintenance period. Risperidone has the following chemical structure:

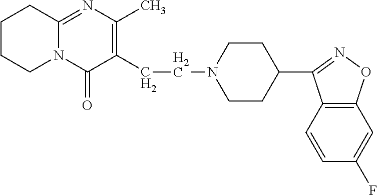 [SMILES: Cc1nc2n(c(=O)c1CCN1CCC(c3noc4cc(F)ccc34)CC1)CCCC2]

After intramuscular or subcutaneous injection, conventional injectable preparations, such as solution, suspension, and emulsion, will be removed from the body quickly. Therefore, frequently parenteral administration is required by treatment of chronic diseases. To solve the foregoing problem, sustained-release microparticles are proposed, which specifically refer to microcapsules prepared by encapsulating drugs into polymers or microsphere disperse systems prepared by dispersing or adsorbing drugs into polymers. Microspheres usually have a size in a unit of μm, so they can be administered to the human body or animals by intramuscular or subcutaneous injection. Microspheres may be prepared according to different drug release rates, so that the drug delivery time can be controlled. Therefore, the effective therapeutic concentration of a drug can be maintained for a long term after single administration, which reduces the total dose of the drug required by treatment to the maximum extent, and improves the patient compliance with drug therapy.

The approved VIVITROL® includes the active ingredient naltrexone, and it is proved that the preparation has a good effect of improving opioid relapse control and quitting alcohol addiction. However, the preparation cannot achieve good results in treatment of novel drug addiction, and especially cannot achieve satisfactory results in treatment of methamphetamine drug addiction.

Currently, there is no report on application of a compound composition of naltrexone and risperidone in addiction treatment.

### SUMMARY

A technical problem to be solved by the present disclosure is to provide a naltrexone sustained-release microsphere preparation capable of effectively prolonging the in vivo action time of a compound of naltrexone and risperidone to reduce the administration frequency of naltrexone and risperidone.

An objective of the present disclosure is to provide a compound sustained-release composition of naltrexone and risperidone, which includes the following components in parts by weight:
5-10 parts of naltrexone sustained-release microspheres, and
1-2 parts of risperidone sustained-release microspheres.

Further, the risperidone sustained-release microspheres are prepared by a W₁/O/W₂ double emulsion-solvent evaporation method.

Further, the naltrexone sustained-release microspheres are prepared by an 01W emulsion-solvent evaporation method.

Further, a preparation method of the risperidone sustained-release microspheres includes the following steps:
preparation of the risperidone sustained-release microspheres by a W₁/O/W₂ double emulsion-solvent evaporation method
S1: dissolving an additive in purified water to form an inner water phase;
S2: dissolving risperidone and a fat-soluble polymer in an organic solvent to form an oil phase;
S3: adding the inner water phase to the oil phase, and performing ultrasonic emulsification to obtain a primary emulsion; and
S4: adding dropwise the primary emulsion to an aqueous solution of a water-soluble polymer, stirring until uniform, evaporating the organic solvent, washing, collecting, and drying to obtain risperidone sustained-release microspheres.

Further, the additive is selected from at least one of sodium chloride, mannitol, disodium hydrogen phosphate, and sodium dihydrogen phosphate.

Further, the fat-soluble polymer is selected from at least one of polylactic acid (PLA), poly(lactic-co-glycolic acid) (PLGA), polycaprolactone (PCL), polycarbonate, polyglycolic acid (PGA), polycyanoacrylate, and polyether ester.

Further, the water-soluble polymer is selected from polyvinyl alcohol.

Further, in the poly(lactic-co-glycolic acid) (PLGA), a ratio of lactic acid to glycolic acid is (1-9):1.

Further, the weight-average molecular weight (M w) of the water-soluble polymer or the fat-soluble polymer is 6,000-220,000 Da, and preferably 50,000-100,000 Da.

Further, the organic solvent is selected from at least one of dichloromethane and ethyl acetate.

Further, in S1, a dosage ratio of the additive to the purified water is 50-300 mg/mL, and preferably 75-150 mg/mL.

Further, in S2, the concentration of risperidone is 100-300 mg/mL, and preferably 100-150 mg/mL; and the concentration of the fat-soluble polymer is 100-300 mg/mL, and preferably 100-150 mg/mL.

Further, in S4, a dosage ratio of the primary emulsion to the aqueous solution of the water-soluble polymer is (1-3): 80, and preferably (2-3): 80.

Further, in S4, in the aqueous solution of the water-soluble polymer, the content of the water-soluble polymer is 0.1-2 wt %.

Further, in S4, the content of the water-soluble polymer is 0.5-0.6 wt %.

Further, the aqueous solution contains 0-5 wt % sodium chloride or 0-10 wt % sucrose.

Further, the stirring is performed at 500-5,000 revolutions/minute, and preferably 700-1,000 revolutions/minute, and the evaporation is performed at 200-3,000 revolutions/minute, preferably 500-1,000 revolutions/minute, at 30-40° C. for 24 h.

Further, a preparation method of the naltrexone sustained-release microspheres includes the following steps:
preparation of naltrexone sustained-release microspheres by an O/W emulsion-solvent evaporation method
S1′: dissolving naltrexone and a fat-soluble polymer in an organic solvent to form an oil phase; and
S2′: adding dropwise the oil phase to an aqueous solution of a water-soluble polymer, stirring until uniform, evaporating the organic solvent, washing, collecting, and drying to obtain naltrexone sustained-release microspheres.

Further, the fat-soluble polymer is selected from at least one of polylactic acid (PLA), poly(lactic-co-glycolic acid) (PLGA), polycaprolactone (PCL), polycarbonate, polyglycolic acid (PGA), polycyanoacrylate, and polyether ester.

Further, the water-soluble polymer is selected from polyvinyl alcohol.

Further, the organic solvent is selected from at least one of dichloromethane and ethyl acetate.

Further, in the poly(lactic-co-glycolic acid) (PLGA), a ratio of lactic acid to glycolic acid is (1-9): 1.

Further, the weight-average molecular weight (M w) of the water-soluble polymer or the fat-soluble polymer is 6,000-220,000 Da, and preferably 50,000-100,000 Da.

Further, in S1′, the concentration of naltrexone is 50-300 mg/mL, and preferably 100-150 mg/mL; and the concentration of the fat-soluble polymer is 100-300 mg/mL, and preferably 100-150 mg/mL.

Further, in S2′, a dosage ratio of the oil phase to the aqueous solution of the water-soluble polymer is (1-3): 80, and preferably (2-3): 80.

Further, in S2′, in the aqueous solution of the water-soluble polymer, the content of the water-soluble polymer is 0.1-2 wt %, and preferably 0.5-1 wt %.

Further, the aqueous solution contains 0-10 wt % sodium chloride or 0-20 wt % sucrose, preferably 8-10 wt % sucrose.

Further, the stirring is performed at 500-5,000 revolutions/minute, and preferably 700-1,000 revolutions/minute, and the evaporation is performed at 200-3,000 revolutions/minute, preferably 500-1,000 revolutions/minute, at 30-40° C. for 24 h.

Another objective of the present disclosure is to provide a preparation including the composition, which is prepared by uniformly mixing the composition with a lubricant, compressing into tablets, and coating.

Further, the lubricant is selected from at least one of sodium stearyl fumarate, magnesium stearate, and a stearic acid.

Further, a dosage of lubricant is 0.05-0.1 wt % of the composition.

Still another objective of the present disclosure is to provide application of the composition and the preparation, which is application in preparation of a product for treating or relieving amphetamine, ketamine, cocaine or cannabis addiction.

The present disclosure has the following advantages.

Patients with opioid or drug addiction usually develop psychiatric symptoms in addition to addiction symptoms. Therefore, during treatment of patients with opioid or drug addiction, ancillary interventions for mental stability of the patients are also important. In the present disclosure, naltrexone and risperidone are combined into a compound for use, which further improve the therapeutic effect on patients with opioid or drug addiction.

1. The sustained-release preparation of the present disclosure is composed of naltrexone, risperidone, a biodegradable pharmaceutical polymer material, and an additive.

2. The preparation method of the present disclosure improves the stability of naltrexone and risperidone in the preparation process and release process, is simple and easy to operate, and has good repeatability.

3. The sustained-release microspheres of the present disclosure may be sustainably released in vitro for more than 12 weeks, the release conforms to a zeroth-order approximation mode, and the release rate is stable.

4. The suitable preparation method and parameter control are adopted in the present disclosure, so that naltrexone sustained-release microspheres and risperidone sustained-release microspheres are released synchronously, and an administration period is easier to control to avoid an uncertain administration period due to non-synchronous release of the two drugs.

5. In view of the fact that there is no anti-amphetamine relapse product on the market, the preparation of the present disclosure can better help patients avoid relapse during the window period due to frequent medications by virtue of its sustained-release characteristics.

### BRIEF DESCRIPTION OF THE DRAWINGS

FIG. 1 shows a scanning electron microscopy image of naltrexone sustained-release microspheres prepared in Example 3 of the present disclosure;

FIG. 2 shows a scanning electron microscopy image of risperidone sustained-release microspheres prepared in Example 3 of the present disclosure;

FIG. 3 shows an in vitro cumulative release curve of a compound sustained-release preparation of naltrexone and risperidone prepared in Example 1 of the present disclosure, where the Y-axis represents a cumulative release percentage (%), and the X-axis represents time (day);

FIG. 4 shows an in vitro cumulative release curve of a compound sustained-release preparation of naltrexone and risperidone prepared in Example 2 of the present disclosure, where the Y-axis represents a cumulative release percentage (%), and the X-axis represents time (day);

FIG. 5 shows an in vitro cumulative release curve of a compound sustained-release preparation of naltrexone and risperidone prepared in Example 3 of the present disclosure, where the Y-axis represents a cumulative release percentage (%), and the X-axis represents time (day);

FIG. 6 shows an in vitro cumulative release curve of a compound sustained-release preparation of naltrexone and risperidone prepared in Comparative Example 1 of the present disclosure, where the Y-axis represents a cumulative release percentage (%), and the X-axis represents time (day);

FIG. 7 shows an in vitro cumulative release curve of a compound sustained-release preparation of naltrexone and risperidone prepared in Comparative Example 2 of the present disclosure, where the Y-axis represents a cumulative release percentage (%), and the X-axis represents time (day);

FIG. 8 shows a in vivo drug-time curve of the compound sustained-release preparation of naltrexone and risperidone prepared in Example 1 of the present disclosure, where the Y-axis represents a plasma concentration percentage (%), and the X-axis represents time (day); and

FIG. 9 shows a comparison diagram of sensitization data of animal models according to examples of the present disclosure, where the Y-axis represents a travelled distance (cm), and the X-axis represents time (day).

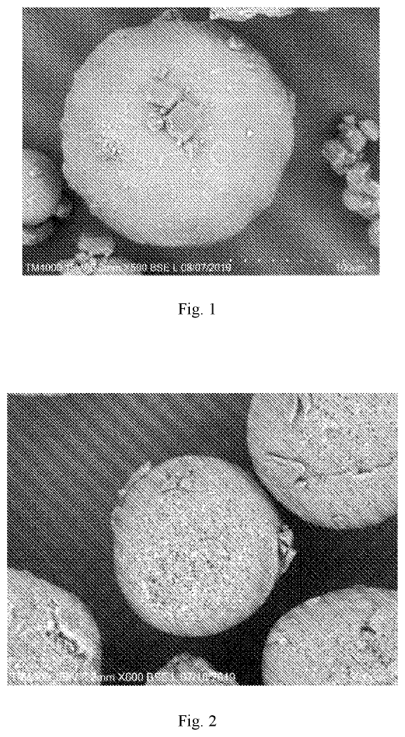

### DETAILED DESCRIPTION OF THE EMBODIMENTS

The present disclosure will be further described below with reference to the drawings.

### Example 1

1. Preparation of Risperidone Microspheres

Preparation of Risperidone Microspheres by a W₁/O/W₂ Double Emulsion-Solvent Evaporation Method

(1) 0.6 g of sodium chloride was dissolved in 3 mL of purified water to form an inner water phase.

(2) 3.5 g of risperidone and 3.5 g of PLA (M_{w} 80,000 Da) were dissolved in dichloromethane to form an oil phase in which the concentration of risperidone was 150 mg/mL and the concentration of PLA was 150 mg/mL.

(3) The inner water phase was added to the oil phase, and the mixture was ultrasonically emulsified to form a primary emulsion.

(4) The primary emulsion was added dropwise to an aqueous solution (containing 0.5% polyvinyl alcohol and 5% sodium chloride) according to a ratio of 1:40, the mixture was thoroughly stirred at 1,000 revolutions/minute until uniform, the organic solvent was evaporated by stirring at 800 revolutions/minute at 35° C. for 24 h to obtain sustained-release microspheres, and the microspheres were washed, collected, and dried.

2. Preparation of Naltrexone Microspheres

Preparation of Naltrexone Microspheres by an O/W Emulsion-Solvent Evaporation Method

(1) 3.5 g of naltrexone and 3.5 g of PLA (M w 80,000 Da) were dissolved in dichloromethane to form an oil phase in which the concentration of naltrexone was 150 mg/mL and the concentration of PLA was 150 mg/mL.

(2) The oil phase was added dropwise to an aqueous solution (containing 0.5% polyvinyl alcohol and 10% sucrose) according to a ratio of 1:40, the mixture was thoroughly stirred at 1,000 revolutions/minute until uniform, the organic solvent was evaporated by stirring at 800 revolutions/minute at 35° C. for 24 h to obtain sustained-release microspheres, and the microspheres were collected and dried.

3. Mixing and Compression

10 parts of prepared naltrexone microspheres, 1 part of prepared risperidone microspheres, and 0.05% magnesium stearate were mixed and compressed into tablets.

4. The tablets were coated by spraying to obtain a compound sustained-release preparation of naltrexone and risperidone.

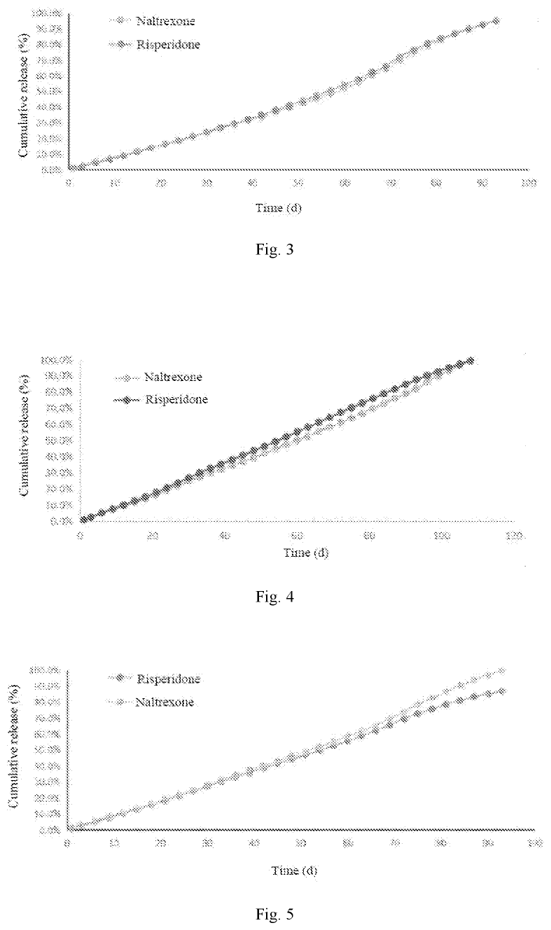

An in vitro cumulative release curve of the compound sustained-release preparation of naltrexone and risperidone prepared in this example is shown in FIG. 3, and it can be seen that the in vitro cumulative release period exceeds 90 days, and naltrexone and risperidone are released synchronously.

### Example 2

1. Preparation of Risperidone Microspheres

Preparation of Risperidone Microspheres by a W₁/O/W₂ Double Emulsion-Solvent Evaporation Method

(1) 0.3 g of sodium dihydrogen phosphate was dissolved in 2 mL of water to form an inner water phase.

(2) 4.0 g of risperidone and 6.0 g of PLGA (lactic acid: glycolic acid=90:10, M_{w} 50,000 Da) were dissolved in dichloromethane to form an oil phase in which the concentration of risperidone was 100 mg/mL and the concentration of PLGA was 150 mg/mL.

(3) The inner water phase was added to the oil phase, and the mixture was ultrasonically emulsified to form a primary emulsion.

(4) The primary emulsion was added dropwise to an aqueous solution (containing polyvinyl alcohol and 5% sodium chloride) according to a ratio of 3:80, the mixture was thoroughly stirred at 700 revolutions/minute until uniform, the organic solvent was evaporated by stirring at 1,000 revolutions/minute at 35° C. for 24 h to obtain sustained-release microspheres, and the microspheres were washed, collected, and dried.

2. Preparation of Naltrexone Microspheres

Preparation of Naltrexone Microspheres by an O/W Emulsion-Solvent Evaporation Method

(1) 4.5 g of naltrexone and 6 g of PLGA (lactic acid: glycolic acid=90:10, M w Da) were dissolved in dichloromethane to form an oil phase in which the concentration of naltrexone was 150 mg/mL and the concentration of PLGA was 200 mg/mL.

(2) The oil phase was added dropwise to an aqueous solution (containing 1.0% polyvinyl alcohol and 8% sucrose) according to a ratio of 3:80, the mixture was thoroughly stirred at 700 revolutions/minute until uniform, the organic solvent was evaporated by stirring at 1,000 revolutions/minute at 35° C. for 24 h to obtain sustained-release microspheres, and the microspheres were collected and dried.

3. Mixing and Compression

5 parts of prepared naltrexone microspheres, 1 part of prepared risperidone microspheres, and 0.1% sodium stearyl fumarate were mixed and compressed into tablets.

4. The tablets were coated by spraying to obtain a compound sustained-release preparation of naltrexone and risperidone.

An in vitro cumulative release curve of the compound sustained-release preparation of naltrexone and risperidone prepared in this example is shown in FIG. 4, and it can be seen that the in vitro cumulative release period exceeds 90 days, and naltrexone and risperidone are released synchronously.

### Example 3

1. Preparation of Risperidone Microspheres

Preparation of Risperidone Microspheres by a W₁/O/W₂ Double Emulsion-Solvent Evaporation Method

(1) 0.5 g of disodium hydrogen phosphate was dissolved in 5 mL of water to form an inner water phase.

(2) 5.0 g of risperidone and 6.0 g of PLGA (lactic acid: glycolic acid=50:50, M_{w} 100,000 Da) were dissolved in ethyl acetate to form an oil phase in which the concentration of risperidone was 100 mg/mL and the concentration of PLGA was 120 mg/mL.

(3) The inner water phase was added to the oil phase, and the mixture was ultrasonically emulsified to form a primary emulsion.

(4) The primary emulsion was added dropwise to an aqueous solution (containing polyvinyl alcohol and 15% sucrose) according to a ratio of 1:40, the mixture was thoroughly stirred at 800 revolutions/minute until uniform, the organic solvent was evaporated by stirring at 700 revolutions/minute at 35° C. for 24 h to obtain sustained-release microspheres, and the microspheres were washed, collected, and dried.

2. Preparation of Naltrexone Microspheres

Preparation of Naltrexone Microspheres by an O/W Emulsion-Solvent Evaporation Method

(1) 5 g of naltrexone and 5 g of PLGA (50:50, M_{w} 100,000 Da) were dissolved in dichloromethane to form an oil phase in which the concentration of naltrexone was 100 mg/mL and the concentration of PLGA was 100 mg/mL.

(2) The oil phase was added dropwise to an aqueous solution (containing 0.7% polyvinyl alcohol and 10% sucrose) according to a ratio of 1:40, the mixture was thoroughly stirred at 900 revolutions/minute until uniform, the organic solvent was evaporated by stirring at 700 revolutions/minute at 35° C. for 24 h to obtain sustained-release microspheres, and the microspheres were collected and dried.

3. Mixing and Compression

5 parts of prepared naltrexone microspheres, 2 parts of prepared risperidone microspheres, 0.025% sodium stearyl fumarate, and 0.025% magnesium stearate were mixed and compressed into tablets.

4. The tablets were coated by spraying to obtain a compound sustained-release preparation of naltrexone and risperidone.

An in vitro cumulative release curve of the compound sustained-release preparation of naltrexone and risperidone prepared in this example is shown in FIG. 5, and it can be seen that the in vitro cumulative release period exceeds 90 days, and naltrexone and risperidone are released synchronously.

### Comparative Example 1 Preparation of Risperidone Microspheres by an O/W Emulsion-Solvent Evaporation Method Different from Example 1

1. Preparation of Risperidone Microspheres

Preparation of Risperidone Microspheres by an O/W Emulsion-Solvent Evaporation Method

(1) 3 g of risperidone and 3 g of PLA (M w 80,000 Da) were dissolved in dichloromethane to form an oil phase in which the concentration of risperidone was 150 mg/mL and the concentration of PLA was 150 mg/mL.

(2) The oil phase was added dropwise to an aqueous solution (containing 0.5% polyvinyl alcohol and 10% sucrose) according to a ratio of 1:40, the mixture was thoroughly stirred at 1,000 revolutions/minute until uniform, the organic solvent was evaporated by stirring at 800 revolutions/minute at 35° C. for 24 h to obtain sustained-release microspheres, and the microspheres were washed, collected, and dried.

2. Preparation of Naltrexone Microspheres

Preparation of Naltrexone Microspheres by an O/W Emulsion-Solvent Evaporation Method

(1) 3 g of naltrexone and 3 g of PLA (M w 80,000 Da) were dissolved in dichloromethane to form an oil phase in which the concentration of naltrexone was 150 mg/mL and the concentration of PLA was 150 mg/mL.

(2) The oil phase was added dropwise to an aqueous solution (containing 0.5% polyvinyl alcohol and 10% sucrose) according to a ratio of 1:40, the mixture was thoroughly stirred at 1,000 revolutions/minute until uniform, the organic solvent was evaporated by stirring at 800 revolutions/minute at 35° C. for 24 h to obtain sustained-release microspheres, and the microspheres were collected and dried.

3. Mixing and Compression

10 parts of prepared naltrexone microspheres, 1 part of prepared risperidone microspheres, and 0.05% magnesium stearate were mixed and compressed into tablets.

4. The tablets were coated by spraying to obtain a compound sustained-release preparation of naltrexone and risperidone.

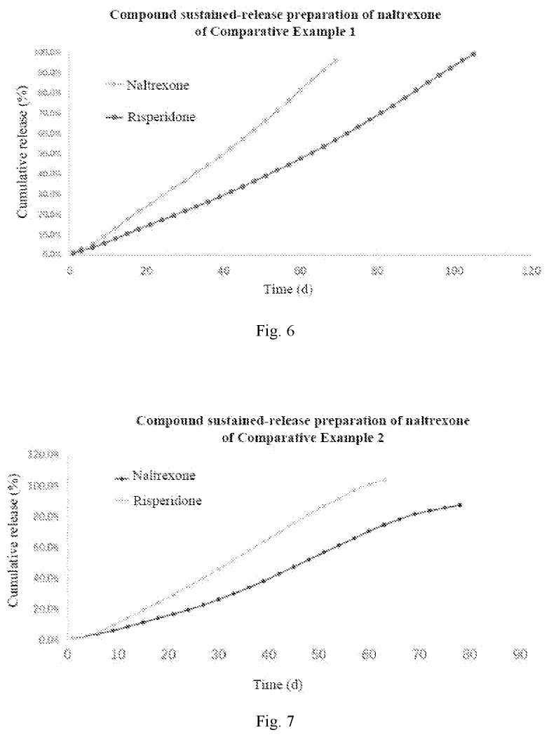

An in vitro cumulative release curve of the compound sustained-release preparation of naltrexone and risperidone prepared in this Comparative Example is shown in FIG. 6, and it can be seen that the in vitro cumulative release period is only 70 days, and naltrexone and risperidone cannot be released synchronously.

### Comparative Example 2 Preparation of Naltrexone Microspheres by a W₁/O/W₂ Double Emulsion-Solvent Evaporation Method Different from Example 1

1. Preparation of Risperidone Microspheres

Preparation of Risperidone Microspheres by a W₁/O/W₂ Double Emulsion-Solvent Evaporation Method

(1) 0.6 g of sodium chloride was dissolved in 3 mL of purified water to form an inner water phase.

(2) 3.5 g of risperidone and 3.5 g of PLA (M w 80,000 Da) were dissolved in dichloromethane to form an oil phase in which the concentration of risperidone was 150 mg/mL and the concentration of PLA was 150 mg/mL.

(3) The inner water phase was added to the oil phase, and the mixture was ultrasonically emulsified to form a primary emulsion.

(4) The primary emulsion was added dropwise to an aqueous solution (containing polyvinyl alcohol and 5% sodium chloride) according to a ratio of 1:40, the mixture was thoroughly stirred at 1,000 revolutions/minute until uniform, the organic solvent was evaporated by stirring at 800 revolutions/minute at 35° C. for 24 h to obtain sustained-release microspheres, and the microspheres were collected and dried.

2. Preparation of Naltrexone Microspheres

Preparation of Naltrexone Microspheres by a W₁/O/W₂ Double Emulsion-Solvent Evaporation Method

(1) 0.6 g of sodium chloride was dissolved in 4 mL of purified water to form an inner water phase.

(2) 3.5 g of naltrexone and 3.5 g of PLA (M_{w} 80,000 Da) were dissolved in dichloromethane to form an oil phase in which the concentration of naltrexone was 150 mg/mL and the concentration of PLA was 150 mg/mL.

(3) The inner water phase was added to the oil phase, and the mixture was ultrasonically emulsified to form a primary emulsion.

(4) The primary emulsion was added dropwise to an aqueous solution (containing polyvinyl alcohol and 10% sucrose) according to a ratio of 1:40, the mixture was thoroughly stirred at 1,000 revolutions/minute until uniform, the organic solvent was evaporated by stirring at 800 revolutions/minute at 35° C. for 24 h to obtain sustained-release microspheres, and the microspheres were collected and dried.

3. Mixing and Compression

10 parts of prepared naltrexone microspheres, 1 part of prepared risperidone microspheres, and 0.05% magnesium stearate were mixed and compressed into tablets.

4. The tablets were coated by spraying to obtain a compound sustained-release preparation of naltrexone and risperidone.

An in vitro cumulative release curve of the compound sustained-release preparation of naltrexone and risperidone prepared in this Comparative Example is shown in FIG. 7, and it can be seen that the release periods of naltrexone and risperidone are less than 80 days, and naltrexone and risperidone cannot be released synchronously.

It can be seen from Example 1 and Comparative Example 1 that in a case that the preparation methods of risperidone microspheres are different and the preparation methods of naltrexone microspheres are the same, the release behaviors of risperidone are similar, and the release behaviors of naltrexone are greatly different. Moreover, it can be seen from Example 1 and Comparative Example 2 that in a case that the preparation methods of risperidone microspheres are the same and the preparation methods of naltrexone microspheres are different, the release behaviors of naltrexone are similar, and the release behaviors of risperidone are greatly different. It shows the complexity of the release behaviors of the two sustained-release microspheres of the present disclosure when mixed, and to achieve the synchronous release of the two sustained-release microspheres, the parameters need to be limited within the ranges required by the present disclosure.

### Comparative Example 3 Preparation by Using a Fat-Soluble Polymer with a Molecular Weight Different from Example 1

1. Preparation of Risperidone Microspheres

Preparation of Risperidone Microspheres by a W₁/O/W₂ Double Emulsion-Solvent Evaporation Method

(1) 0.6 g of sodium chloride was dissolved in 3 mL of purified water to form an inner water phase.

(2) 3.5 g of risperidone and 3.5 g of PLA (M_{w} 5,000 Da) were dissolved in dichloromethane to form an oil phase in which the concentration of risperidone was 150 mg/mL and the concentration of PLA was 150 mg/mL.

(3) The inner water phase was added to the oil phase, and the mixture was ultrasonically emulsified to form a primary emulsion.

(4) The primary emulsion was added dropwise to an aqueous solution (containing 0.5% polyvinyl alcohol and 5% sodium chloride) according to a ratio of 1:40, the mixture was thoroughly stirred at 1,000 revolutions/minute until uniform, the organic solvent was evaporated by stirring at 800 revolutions/minute at 35° C. for 24 h to obtain sustained-release microspheres, and the microspheres were collected and dried.

2. Preparation of Naltrexone Microspheres

Preparation of Naltrexone Microspheres by an O/W Emulsion-Solvent Evaporation Method

(1) 3.5 g of naltrexone and 3.5 g of PLA (M_{w} 5,000 Da) were dissolved in dichloromethane to form an oil phase in which the concentration of naltrexone was 150 mg/mL and the concentration of PLA was 150 mg/mL.

(2) The oil phase was added dropwise to an aqueous solution (containing 0.5% polyvinyl alcohol and 10% sucrose) according to a ratio of 1:40, the mixture was thoroughly stirred at 1,000 revolutions/minute until uniform, the organic solvent was evaporated by stirring at 800 revolutions/minute at 35° C. for 24 h to obtain sustained-release microspheres, and the microspheres were collected and dried.

3. Mixing and Compression

10 parts of prepared naltrexone microspheres, 1 part of prepared risperidone microspheres, and 0.05% magnesium stearate were mixed and compressed into tablets.

4. The tablets were coated by spraying to obtain a compound sustained-release preparation of naltrexone and risperidone.

In this case, naltrexone and risperidone cannot be released synchronously.

Example In Vitro Release

An appropriate amount of naltrexone reference was taken and accurately weighed, a release medium was added to dissolve the naltrexone reference and quantitatively dilute to obtain a solution containing about 50 μg/mL naltrexone reference, and the solution was taken as a naltrexone reference solution.

An appropriate amount of risperidone reference was taken and accurately weighed, a release medium was added to dissolve the risperidone reference and quantitatively dilute to obtain a solution containing about 50 μg/mL risperidone control, and the solution was taken as a risperidone reference solution.

The product of the present disclosure was taken and placed into six 100 mL conical flasks with stoppers, 80 mL of 0.1 M anhydrous dipotassium hydrogen phosphate solution (a solution prepared by accurately sucking and placing 25 mL of 1 M sodium hydroxide solution into a 1,000 mL volumetric flask, adding a 0.1 M anhydrous dipotassium hydrogen phosphate solution for dilution, and regulating the pH value of the diluted solution to 11.2±0.05) was used as a solvent, and the conical flasks were tightly covered with the stoppers and shaken in a water bath oscillator at 100 times/minute at 37±0.5° C. The medium was replaced on day 1, day 3, day 6, day 9, day 12, day 15, . . . , and day 120, the solution was filtered, a subsequent filtrate was taken as a test solution, and the release medium was replenished in the operating container in time. The test solution was detected by high-performance liquid chromatography (a high-performance liquid chromatography method described in General Rule 0512 of Chinese Pharmacopoeia 2020) at a wavelength of 280 nm.

In vitro cumulative release results are shown in FIG. 3 to FIG. 7, and it can be seen that the release period of the product of the present disclosure is more than 12 weeks, and the naltrexone and risperidone drugs are released synchronously. However, the two drugs cannot be released synchronously in Comparative Examples, and a difference between release end points is more than 2 weeks.

Example Pharmacokinetics in Animals

SD rats, half male and half female, were taken as experimental animals. The naltrexone compound preparation sample of Example 1 was used. The compound sustained-release preparation was implanted into the back of each rat, and blood of each rat was collected at set time points for analysis of drug-time data.

Blood collection method: the rat was anesthetized with isoflurane, about 1 mL of blood was collected from the retroorbital venous plexus, the collected whole blood was placed into an EP tube containing heparin immediately, the EP tube was shaken by reversing top and bottom three times, and placed in crushed ice, and centrifugation was performed (at 4,000 rpm at 4° C. for about 5 min) within 1 h, and the centrifuged plasma sample was stored below −20° C. Naltrexone and its metabolite 6β-naltrexol, and risperidone and its metabolite 9-hydroxyrisperidone in the blood sample were detected by LC-MS/MS. Blood was collected for analysis once a week at time points 0 h, 4 h, D1, D3, D7, D10, D16, D39, D45, D60, D75, and D90.

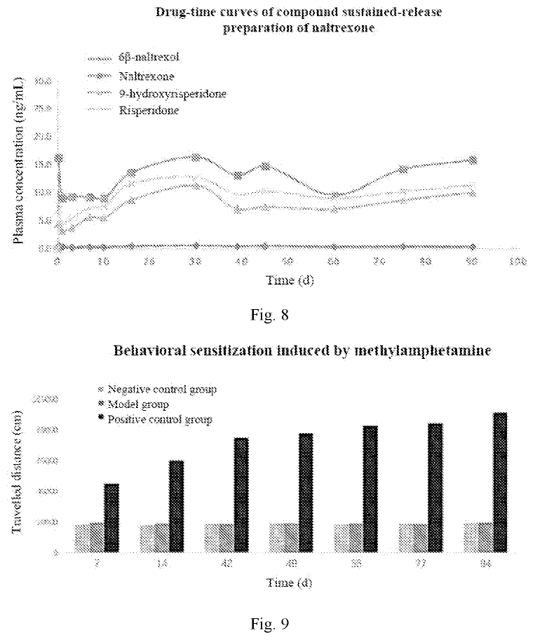

Drug-time analysis results are shown in FIG. 8, and it can be seen that the product of the present disclosure can effectively release risperidone and naltrexone into the blood in the body of the experimental animal, and the plasma concentration is kept stable within 90 days.

Example Animal Pharmacodynamic Evaluation

All mice were bred under the same conditions. The product of the present disclosure was surgically implanted into each mouse in a model group before an experiment. Except implantation of the product of the present disclosure, other experimental conditions of a positive control group were the same as those of the model group. Mice in a negative control group were normally bred without undergoing experimental treatment. Behavioral differences among the model group, the positive control group, and the negative control group were observed during the experiment.

36 SD mice weighing 120-150 g, half male and half female, were taken. The mice were randomly divided into 3 groups: a positive control group, a negative control group, and a model group. The compound sustained-release preparation (prepared in Example 1) was implanted into the back of each mouse in the model group before administration, and a challenge experiment was carried out on the second day after surgery.

During the formative period, methylamphetamine (2 mg/kg) was daily intraperitoneally injected into each mouse in the positive control group and the model group, an equal amount of normal saline was intraperitoneally injected into each mouse in the negative control group for 7 consecutive days. During the conversion period, the drug was withdrawn for 7 days without any treatment. The expression period lasted for 12 weeks, during the expression period, methylamphetamine (2 mg/kg) was intraperitoneally injected into each mouse every 7 days for stimulation, and spontaneous activities of the mice were observed 30 minutes after injection.

Results are shown in FIG. 9, and it can be seen that from day 7, activities of the mice in the positive control group are significantly increased (P<0.05), which indicates that spontaneous activities of the mice chronically administered with methylamphetamine are obvious, and methylamphetamine can induce behavioral sensitization. Compared with the positive control group, spontaneous activities of the mice in the model group are significantly reduced (P<0.01) on day 7, day 14, day 42, day 49, day 56, day 77, and day 84, which are similar to those of the mice in the negative control group. Compared with the mice in the model group on day 7, spontaneous activities of the mice in the model group on day 14, day 42, day 49, day 56, day 77, and day 84 are not gradually increased as those of the mice in the positive control group, which indicates that after the pre-implantation of the compound sustained-release preparation of naltrexone and risperidone, the mice are antagonistic to methamphetamine, and the body is protected.

Finally, it should be noted that the above content is only used to describe the technical solutions of the present disclosure, rather than to limit the scope of protection of the present disclosure. Simple modifications or equivalent replacements made by those of ordinary skill in the art to the technical solutions of the present disclosure will not depart from the essence and scope of the technical solutions of the present disclosure.

1. A composition, comprising the following components in parts by weight:
5-10 parts of naltrexone present in sustained-release microspheres; and
1-2 parts of risperidone present in sustained-release microspheres,
wherein the sustained-release microspheres of the risperidone are being prepared by a double emulsion-solvent evaporation method, and the risperidone sustained-release microspheres of the naltrexone are prepared by an emulsion-solvent evaporation method.

2. (canceled)

3. The composition according to claim 1, wherein the double emulsion-solvent evaporation method comprises:
dissolving an additive in water to form an inner water phase;
dissolving risperidone and a fat-soluble polymer in an organic solvent to form an oil phase;
adding the inner water phase to the oil phase, and performing ultrasonic emulsification to obtain a primary emulsion; and
adding the primary emulsion to an aqueous solution comprising a water-soluble polymer, and evaporating the organic solvent, collecting.

4. The composition according to claim 3, wherein
the additive is dissolved in the water to reach a concentration of 50-300 mg/mL;
the risperidone is dissolved in the organic solvent to reach a concentration of 100-300 mg/mL, the organic solvent is selected from the group consisting of dichloromethane, ethyl acetate and the combination thereof, and the fat-soluble polymer is dissolved in the organic solvent to reach a concentration of 100-300 mg/mL; and
the primary emulsion is added to the aqueous solution at a volume ratio of (1-3):80, and the water-soluble polymer is present in the aqueous solution at a concentration of 0.1-2 wt %.

5. The composition according to claim 4, wherein the aqueous solution further comprises sodium chloride at a concentration of ≤5 wt % or sucrose at a concentration of ≤15 wt %.

6. (canceled)

7. The composition according to claim 1, wherein the emulsion-solvent evaporation method comprises:
dissolving naltrexone and a second fat-soluble polymer in a second organic solvent to form a second oil phase; and
adding, dropwise, the second oil phase to a second aqueous solution comprising a second water-soluble polymer, and evaporating the second organic solvent.

8. The composition according to claim 7, wherein
the naltrexone is dissolved in the second organic solvent to reach a concentration of 50-300 mg/mL, the fat-soluble polymer is dissolved in the second organic solvent to reach a concentration of 100-300 mg/mL, and the second organic solvent is selected from the group consisting of dichloromethane, ethyl acetate, and the combination thereof; and
the second oil phase is added to the second aqueous solution at a volume ratio of (1-3):80, the water-soluble polymer is present in the second aqueous solution at a concentration of 0.1-2 wt % and the second aqueous solution further comprises 0-10 wt % sodium chloride or 0-20 wt % sucrose.

9. The composition according to claim 1, which further comprises a lubricant, and is provided as a tablet.

10. A method for treating or relieving an addiction of amphetamine, ketamine, cocaine or cannabis in a subject, comprising administering to the subject the composition of claim 1.

11. The composition according to claim 3, wherein the additive is selected from the group consisting of sodium chloride, mannitol, disodium hydrogen phosphate, sodium dihydrogen phosphate, and combinations thereof.

12. The composition according to claim 3, wherein the fat-soluble polymer is selected from the group consisting of polylactic acid, poly(lactic-co-glycolic acid), polycaprolactone, polycarbonate, polyglycolic acid, polycyanoacrylate, polyether ester, and combinations thereof.

13. The composition according to claim 3, wherein the water-soluble polymer is a polyvinyl alcohol.

14. The composition according to claim 4, wherein the water-soluble polymer is present in the aqueous solution at a concentration of 0.5-0.6 wt %.

15. The composition according to claim 7, wherein the fat-soluble polymer is selected from the group consisting of polylactic acid, poly(lactic-co-glycolic acid), polycaprolactone, polycarbonate, polyglycolic acid, polycyanoacrylate, polyether ester, and combinations thereof.

16. The composition according to claim 7, wherein the water-soluble polymer is a polyvinyl alcohol.

17. The composition according to claim 8, wherein the water-soluble polymer is present in the second aqueous solution at a concentration of 0.5-1 wt %.

18. The composition according to claim 9, wherein the lubricant is selected from the group consisting of sodium stearyl fumarate, magnesium stearate, a stearic acid, and combinations thereof.
